(19) United States
(12) Patent Application Publication
(10) Pub. No.: US 2005/0106110 A1
(43) Pub. Date: May 19, 2005

(54) Stabilized vaterite

(76) Inventor: Sung-Tsuen Liu, Aberdeen, MD (US)

Correspondence Address: Carlos Nieves, Esq.;J. M. Huber Corporation, 333 Thornall Street, Edison, NJ, 08837-2220, US

(21) Appl. No.: 10/717,310

(22) Filed: Nov. 19, 2003

(51) Int. Cl. C01F 5/24; C01F 11/18; A61K 7/16

(52) U.S. Cl. 424/49; 423/430

(57) ABSTRACT

A precipitated calcium carbonate provides excellent cleaning properties without being excessively abrasive. The precipitated calcium carbonate has a vaterite crystalline form, a primary particle size of about 0.2 μm to about 3 μm, and an aggregate particle size of less than about 4 μm.

### BACKGROUND OF THE INVENTION

Precipitated calcium carbonates find use in a broad range of manufactured and consumer products ranging from paper and paper coatings to plastic and elastomer reinforcement to food supplements and cosmetics. Calcium carbonates are particularly useful in dentifrice products (such as toothpastes) where they function as abrasives. Because of this functional versatility, and also because calcium carbonates, when compared to other dentifrice abrasives, such as silica and dicalcium phosphate, are much less expensive, there is a strong desire among toothpaste and dentifrice formulators to include them in their products.

Precipitated calcium carbonate exists in three primary crystalline forms: calcite, aragonite and vaterite. Many morphological shapes exist for these crystalline forms. Calcite is trigonal with typical crystal habits such as scalenohedron, rhombohedron, hexagonal prism, and pinacoid, cubic, and prismatic; aragonite is orthorhombic with typical crystal habits of twinned hexagonal prismatic crystals, as well as a diverse assortment of thin elongated prismatic, curved bladed, steep pyramidal (spiked) and chisel shaped crystals, branching tree, coral or worm-like delicate form called flosferri; and vaterite is hexagonal with typically a spherical crystal habit. In nature, calcite is the stable calcium carbonate form with aragonite being technically unstable at normal surface temperatures and pressures and vaterite being unstable, converting readily to calcite and usually losing its spherical shape.

As mentioned above, precipitated calcium carbonates offer certain advantages mentioned above, like functional versatility and lower cost, but they also have certain disadvantages that make them undesirable to most dentifrice formulators. For example, when incorporated into dentifrices most precipitated calcium carbonates had either poor cleaning ability (characteristics of small, scalenohedral-shaped calcium carbonate particles) or were excessively abrasive (characteristic of the large rhombohedral-shaped or spherical-shaped vaterite particles). Thus, in order to provide dental hygiene benefits and be suitable for inclusion in a dentifrice, the calcium carbonate must be sufficiently abrasive to provide cleaning of the tooth surfaces, but simultaneously not so excessively abrasive as to damage the tooth surfaces when incorporated into a dentifrice.

Of the known polymorphs and crystalline forms that precipitated calcium carbonate occurs in, spherical-shaped vaterite particles are the most suitable for use in dentifrice formulations. Unfortunately, these vaterite particles are not stable, particularly in aqueous solutions. When in an aqueous solution the vaterite tends to convert to stable calcite by a recrystallization process. This recrystallization process can reach completion in a relatively short time, from about one hour to about several hours. This poses an extremely difficult problem for preparing spherical vaterite, because even before the completion of manufacture a significant amount of vaterite may have recystallized into calcite.

It has previously been believed that vaterite can be stabilized when it is precipitated in the presence of a “stabilizing agent” such as polysulfonates, polyphosphates, metal salt chelating agents, and certain organic solvents such as methanol. (In actual use, the greater portion of the stabilizing agent is added before or during precipitation). However, such stabilization techniques are not well understood. Moreover, while the spherical-shaped vaterite particles are more suitable than other calcium carbonate particles for dentifrice applications, they are often inadequate when compared with other possible dentifrice additives.

Given the foregoing, there is a need for a precipitated calcium carbonate that provides excellent abrasive performance in dentifrice formulations, as well as techniques to stabilize these precipitated calcium carbonates when part of an aqueous composition.

### BRIEF SUMMARY OF THE INVENTION

The invention includes a vaterite calcium carbonate having a primary particle size of about 0.2 μm to about 3 μm and an aggregate particle size of less than about 4 μm.

The invention also includes a method for forming calcium carbonate comprising the steps of: preparing a calcium chloride-monoethanolamine solution; (b) introducing carbon dioxide into the calcium chloride-monoethanolamine solution to form spherical vaterite calcium carbonate by a precipitation reaction and (c) adding a stabilizing agent to the already formed spherical vaterite calcium carbonate.

The invention also includes a method for forming vaterite calcium carbonate comprising the steps of: forming vaterite calcium carbonate under high shear conditions; and adding a stabilizing agent to the already formed spherical vaterite calcium carbonate.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIG. 1 is a SEM photomicrograph of Example 1 spherical vaterite showing a primary particles size of about 0.35 to 1.5 μm and an aggregate particle size of about 1.3 μm.

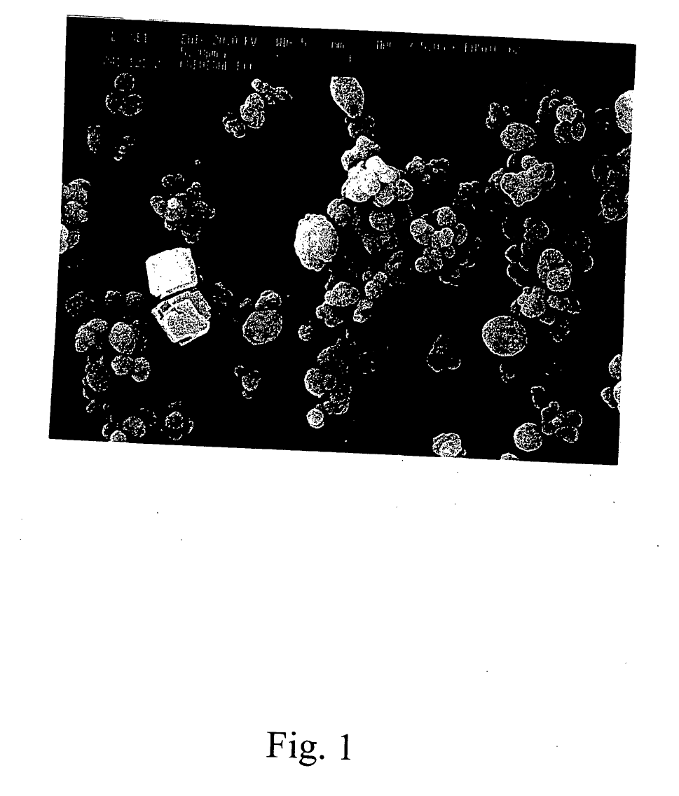

FIG. 2 is a SEM photomicrograph of Example 2 spherical vaterite showing a primary particle size of about 1.5 μm and an aggregate particle size of about 1.3 μm.

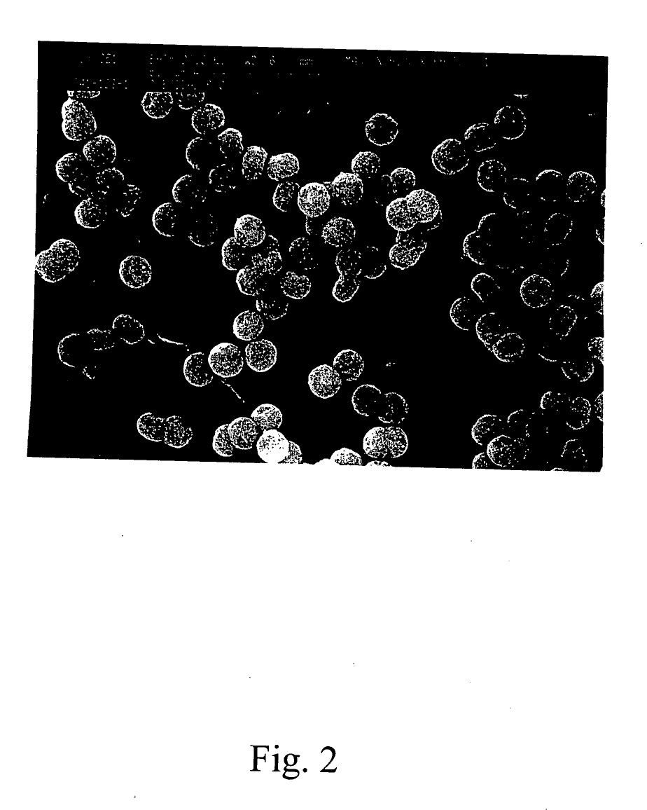

FIG. 3 is a SEM photomicrograph of Comparative Example 3 spherical vaterite showing a primary particle size of about 4.2 to 6.3 μm.

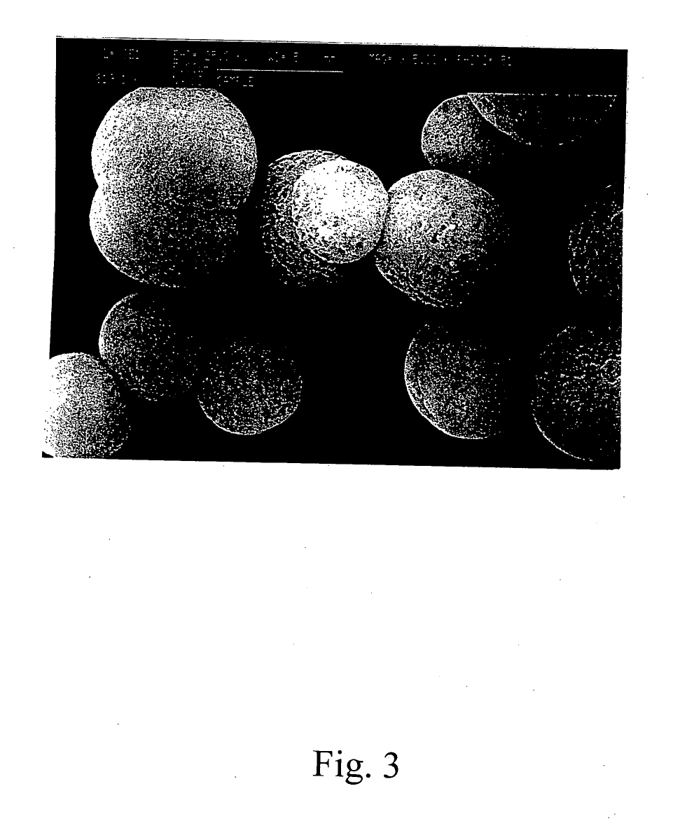

### DETAILED DESCRIPTION OF THE INVENTION

All parts, percentages and ratios used herein are expressed by weight unless otherwise specified. All documents cited herein are incorporated by reference. The following describes preferred embodiments of the present invention, which provides precipitated spherical vaterite calcium carbonate for use in dentifrices, such as toothpastes. While the optimal use for this calcium carbonate is in dentifrices, this calcium carbonate may also be used in a variety of other consumer products.

By “dentifrice” it is meant oral care products such as, without intending to be limiting, toothpastes, tooth powders, chewing gums and denture creams.

By “viscosity build” it is meant increasing dentifrice viscosity as measured by a Brookfield viscometer and is expressed in centipoise (cps).

By “primary particle size” it is meant the size of individual particles, which may be bound to each other to form aggregates, as estimated from visual inspection of a SEM photomicrograph.

By “particle aggregate size” it is meant a group of primary particles bound to each other.

By “particle agglomerate size” it is meant a group of aggregates loosely bound to each other that may be separated by ordinary comminution.

The present invention relates to crystalline precipitated calcium carbonate compositions, known as vaterite, or CaCO₃, which impart improved cleaning and abrasive characteristics when included within a toothpaste or dentifrice. Because they have a unique combination of low abrasiveness and a high degree cleaning ability, the calcium carbonates of the present invention are particularly useful for formulating highly loaded, low-cost toothpaste.

To ensure good cleaning performance a sufficient amount of abrasive calcium carbonate should be added to a toothpaste composition so that the radioactive dentin abrasion (“RDA”) value of the toothpaste is between about 30 and 200. At a RDA of less than 30, the cleaning benefits of the toothpaste will be minimal, while at a RDA of greater than 200, there is serious risk that the toothpaste will be so abrasive that it may damage the tooth dentin along the gum line. Most commercial toothpaste products today have a RDA in the range of 50 to 150, with the average being exactly in the middle around 100. Preferably, the dentifrice should have a RDA value of at least about 30, such as between 30 and 150, most preferably between 30 and 70.

Toothpaste's ability to clean teeth is measured by the pellicle cleaning ratio or “PCR” test, which is described below. A toothpaste formulator's objective is to maximize the PCR value of a toothpaste formulation. Until now, there has been a somewhat linear relationship between RDA and PCR, so that a formulator has to balance the acceptable RDA level with the resulting PCR value.

The RDA and PCR of toothpaste are dependent on the abrasive particle hardness, shape and size and the concentration of the abrasive in the toothpaste. RDA and PCR of the toothpaste and particle abrasivity, measured by an Einlehner method, are described in greater detail below. It has now been discovered that more importantly RDA and PCR are dependent on the abrasive particle shape, primary particle size and the size of the particle aggregates. It is preferred that the calcium carbonate particle shape is essentially spherical with a primary particle size of between about 0.3 μm to 5 μm, such as between 0.3 μm and 2 μm, and an aggregate particle size of between about 1 μm to 10 μm, such as between 1 μm to 2 μm. Preferably the aggregated particle size is less than about 4 μm, such as less than about 3 μm.

Furthermore, by the present invention, abrasive vaterite has been developed that not only has excellent cleaning performance, but shows this cleaning performance at very low abrasiveness. By controlling the batch reaction conditions and adding a FDA sanctioned phosphate to stabilize the precipitated vaterite, an abrasive may be produced that has a small primary particle size and forms small aggregates that can be easily broken apart. When incorporated into a toothpaste composition, the toothpaste provides excellent cleaning benefits, while also being extremely non-abrasive.

The spherical vaterite compositions of the present invention are prepared by forming small, well dispersed primary particles of vaterite. This can be done by using two different types of processes. In the first type, the small, well dispersed primary particles of vaterite are formed by using high shear during the precipitation reaction. In the second type, these particles are formed by using a solvent to keep the formed particles separate. In both cases a small amount of a stabilizing chemical is added after the reaction is complete to keep the formed vaterite from converting to calcite and loosing its spherical shape. These processes will now be described in greater detail.

In the first process, vaterite calcium carbonate is formed under high shear conditions. In this process, sodium carbonate solution is charged into a mixer equipped with high shear mixing means such as a Kady mill, available from Kady International, Scarborough, Me., then a stoichiometric amount of calcium chloride slurry is added all at once. The mixture is stirred at high shear, namely about 10 to about 60 Hz for 5 to 10 minutes, preferably at 30 to 50 Hz to ensure the particles are dispersed. The vaterite slurry is stabilized by the addition of about 0.075% to about 2% stablizing agent, preferably about 1% stabilizing agent such as sodium polyphosphate or tetrasodium etidronate (HEDP). The stabilizing agent should preferably be added in an amount of from about 750 ppm to about 5000 ppm. The vaterite is then filtered and dried by any conventional means, such as oven drying or spray drying.

Alternately, vaterite is produced by reacting calcium chloride with carbon dioxide in the presence of an aged solvent such as monoethanolamine. The high shear employed in the previous process by the Kady mill is not required when vaterite is precipitated in the presence of aged monoethanolamine. This process begins by the preparation of a calcium chloride-monoethanolamine solution by charging calcium chloride and monoethanolamine solutions into a reactor. Preferably the calcium chloride-monoethanolamine solution is aged for more than 1 week, such as 2 weeks. Alternately, fresh monoethanolamine may be used with high shear mixing means. Carbon dioxide gas is introduced into the reactor at a rate to ensure the reaction is completed in about 1 hour, while stirring. Carbonation continues until the mixture reaches about pH 7.5. Then the stirring and carbonation is stopped for about 30 minutes. Afterwards, stirring and carbonation is resumed until the mixture reaches about pH 6.9. The resulting vaterite slurry is filtered and washed until the filtrate conductivity reaches about 500 μS, indicating the wet cake has been essentially washed free of the by-product salt. The stabilizing agent should preferably be added in an amount of from about 750 ppm to about 5000 ppm. The resulting wet cake is stabilized by mixing it with 0.075 to 2% stabilizing agent. The product may then be dried by any conventional means.

This abrasive, precipitated calcium carbonate may then be incorporated into a dentifrice composition, e.g., toothpaste. Typical levels of calcium carbonate abrasives are from about 30 wt % to about 45 wt % of a toothpaste composition, such as from about 40 wt % to about 45 wt %.

In addition to the abrasive component, the dentifrice may also contain several other ingredients such as humectants, thickening agents, (also sometimes known as binders, gums, or stabilizing agents), antibacterial agents, fluorides, condensed phosphates, flavorants, sweeteners, and surfactants.

Humectants serve to add body or “mouth texture” to a dentifrice as well as preventing the dentifrice from drying out. Suitable humectants include polyethylene glycol (at a variety of different molecular weights), propylene glycol, glycerin (glycerol), erythritol, xylitol, sorbitol, mannitol, lactitol, and hydrogenated starch hydrolyzates, as well as mixtures of these compounds. Typical levels of humectants are from about 20 wt % to about 30 wt % of a toothpaste composition.

Thickening agents are useful in the dentifrice compositions of the present invention to provide a gelatinous structure that stabilizes the toothpaste against phase separation. Suitable thickening agents include silica thickener, starch, glycerite of starch, gum karaya (sterculia gum), gum tragacanth, gum arabic, gum ghatti, gum acacia, xanthan gum, guar gum, veegum, carrageenan, sodium alginate, agar-agar, pectin, gelatin, cellulose, cellulose gum, carboxymethyl cellulose, hydroxyethyl cellulose, hydroxypropyl cellulose, hydroxymethyl, hydroxymethyl carboxypropyl cellulose, methyl cellulose, ethyl cellulose, sulfated cellulose, as well as mixtures of these compounds. Typical levels of binders are from about 0 wt % to about 15 wt % of a toothpaste composition.

Antibacterial agents may be included to reduce the presence of microorganisms to below known harmful levels. Suitable antibacterial agents include benzoic acid, sodium benzoate, potassium benzoate boric acid phenolic compounds such as betanapthol, chlorothymol, thymol, anethole, eucalyptol, carvacrol, menthol, phenol, amylphenol, hexylphenol, heptylphenol, octylphenol, hexylresorcinol, laurylpyridinium chloride, myristylpyridinium chloride, cetylpyridinium fluoride, cetylpyridinium chloride, cetylpyridinium bromide. If present, the level of antibacterial agent is preferably from about 0.1 wt % to about 5 wt % of the toothpaste composition.

Sweeteners may be added to the toothpaste composition to impart a pleasing taste to the product. Suitable sweeteners include saccharin (as sodium, potassium or calcium saccharin), cyclamate (as a sodium, potassium or calcium salt), acesulfane-K, thaumatin, neohisperidin dihydrochalcone, ammoniated glycyrrhizin, dextrose, levulose, sucrose, mannose, and glucose.

The toothpaste will also preferably contain fluoride salts to prevent the development and progression of dental caries. Suitable fluoride salts include sodium fluoride, potassium fluoride, zinc fluoride, stannous fluoride, zinc ammonium fluoride, sodium monofluorophosphate, potassium monofluorophosphate, laurylamine hydrofluoride, diethylaminoethyloctoylamide hydrofluoride, didecyldimethylammonium fluoride, cetylpyridinium fluoride, dilaurylmorpholinium fluoride, sarcosine stannous fluoride, glycine potassium fluoride, glycine hydrofluoride, and sodium monofluorophosphate. Typical levels of fluoride salts are from about 0.1 wt % to about 5 wt %.

Condensed phosphates added to the toothpaste may be tetrasodium pyrophosphate, tetrapotassium pyrophosphate, disodium dihydrogen pyrophosphate, trisodium monohydrogen pyrophosphate, pentasodium tripolyphosphate and sodium polymetaphosphate, singly or in combinations thereof.

Surfactants may also be included as additional cleansing and foaming agents, and may be selected from anionic surfactants, zwitterionic surfactants, nonionic surfactants, amphoteric surfactants, and cationic surfactants. Anionic surfactants are preferred, such as metal sulfate salts, such as sodium lauryl sulfate.

The dentifrices disclosed herein may also a variety of additional ingredients such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, coloring and whitening agents and preservatives.

Finally, water provides the balance of the composition in addition to the additives mentioned. The water is preferably deionized and free of impurities. The dentifrice will usually comprise from about 20 wt % to about 35 wt % of water.

The invention will now be described in more detail with respect to the following, specific, non-limiting examples.

### EXAMPLE 1

A stable spherical vaterite calcium carbonate material was prepared in accordance with the present invention as follows. First, 4 liters of 2 M reagent grade sodium carbonate was added to a rotor-stator, high speed dispersion Kady® lab mill (model number L) available from Kady International, Scarborough, Me. Next, 4 liters of 2 M reagent grade calcium chloride was poured into the sodium carbonate solution. The mixture was stirred with the Kady mill at 40 Hz for five minutes. The reaction was carried out at room temperature (25° C.). To the resultant vaterite slurry was added 1.4 g of sodium polyphosphate, (NaPO3)ₙ available from Aldrich Chemical Company. The slurry was placed in a 5-gal. plastic bucket. Three more batches of vaterite were prepared as described above and the slurries were all combined in the bucket. Thereafter, the combined slurry was filtered on a buchner funnel and washed with deionized water. The filter cake was dried in an oven at 100° C. overnight. Physical properties of Example 1 stabilized vaterite is given in Table 1 below.

### EXAMPLE 2

A stable vaterite calcium carbonate was prepared by first preparing a solution of 2 molar calcium chloride and 4 molar monoethanolamine. This solution was stored for 2 weeks before use. 6 liters of the previously prepared calcium chloride/monoethanolamine solution was added to a 5-gal reactor equipped with mechanical stirring means. The pH of this solution was 11.1. Carbon dioxide gas was added to the stirred reactor contents at a rate of 22.5 L/min. Carbonation continued until the reaction mixture reached pH 7.46, which took about 34 minutes. Carbonation and stirring was stopped for 30 minutes. Thereafter, carbonation and stirring resumed until the reaction slurry reached pH 6.87. The resultant vaterite slurry was filtered and washed with deionized water until the filtrate slurry reached a conductivity of 500 μS, indicating the by-product salt had been washed from the wet cake. The vaterite wet cake was stabilized by mixing 12 g of sodium polyphosphate into the wet cake. Thereafter, the resultant stabilized vaterite was dried overnight in an oven at 100° C. Physical properties of Example 2 are given in Table 1 below.

### COMPARATIVE EXAMPLE 3

The procedure of Example 2 was followed, except the solution of 2 M calcium chloride—4 M monoethanolamine was prepared, filtered to remove trace insoluble material, then used without ageing. Physical properties of Comparative Example 3 are given in Table 1 below.

After being prepared as set forth above, several properties of the particulate spherical vaterite, including primary crystal particle size, aggregate particle size and Einlehner abrasion were measured according to the following methods.

Primary particle size was estimated by visually comparing the sizes of spherical particles to a micron marker on a SEM photomicrograph taken at 5×. Individual spherical particles are attached to one another to form aggregates. It is the size of the individual particles that is recorded.

Aggregate particle size (median value) is measured with a Sedigraph 5100 available from Micromeritics Corporation, Norcross, Ga.

The Brass Einlehner (BE) Abrasion value was measured through the use of an Einlehner AT-1000 Abrader. In this test, a Fourdrinier brass wire screen is weighed and exposed to the action of a 10% aqueous calcium carbonate suspension for a fixed number of revolutions, and the amount of abrasion is then determined as milligrams brass lost from the Fourdrinier wire screen per 100,000 revolutions. Disposable supplies required for this test (brass screens, wear plates and PVC tubing) are available from Duncan Associates, Rutland, Vt. and sold as an “Einlehner Test Kit”. Specifically, brass screens (Phosphos Bronze P. M.) were prepared by washing in hot, soapy water (0.5% Alconox) in an ultrasonic bath for 5 minutes, then rinsed in tap water and rinsed again in a beaker containing 150 ml water set in an ultrasonic bath. The screen is rinsed again in tap water, dried in an oven set at 105° C. for 20 minutes, cooled in a desiccator and weighed. Screens were handled with tweezers to prevent skin oils from contaminating the screens. The Einlehner test cylinder is assembled with a wear plate and weighed screen (red line side down—not abraded side) and clamped in place. The wear plate is used for about 25 tests or until worn badly; the weighed screen is used only once.

A 10% calcium carbonate slurry, prepared by mixing 100 g calcium carbonate with 900 g deionized water, was poured into the Einlehner test cylinder. Einlehner PVC tubing was placed onto the agitating shaft. The PVC tubing has 5 numbered positions. For each test, the position of the PVC tubing is incremented until it has been used five times, then discarded. The Einlehner abrasion instrument is re-assembled and the instrument set to run for 87,000 revolutions. Each test takes about 49 minutes. After the cycle is completed, the screen is removed rinsed in tap water, placed in a beaker containing water and set in an ultrasonic bath for 2 minutes, rinsed with deionized water and dried in an oven set at 105° C. for 20 minutes. The dried screen is cooled in a desiccator and reweighed. Two tests are run for each sample and the results are averaged and expressed in mg lost per 100,000 revolutions. The result, measured in units of mg lost per 100,000 revolutions, for a 10% slurry can be characterized as the 10% brass Einlehner (BE) abrasion value.

TABLE 1
Physical Properties

| | Primary | Aggregate | |
|---|---|---|---|
| | Particle Size, | Particle Size, | Brass Einlehner |
| Example | μm | μm | (mg loss) |
| Ex 1 | 0.35-1.5 | 1.3 | 1.85 |
| 841-121-1 | | | |
| Ex 2 | 1.1-1.5 | 1.3 | 1.23 |
| 841-123-1 | | | |
| Comparative Ex. 3 | 4.2-6.3 | 5.6 | 1.5 |
| 819-106 | | | |

### EXAMPLE 4

The procedure of Example 2 was followed, except various amounts of stabilizing chemicals were added to the vaterite wet cake, which was then dried overnight in an oven at 100° C. The dried, stabilized vaterite was then put into water and aged for specified times at 120° F. to simulate stability in toothpaste formulations. Stability of the vaterite was judged by viewing the particle morphology with an optical microscope.

The stabilizing chemicals used were reagent grade sodium polyphosphate, (NaPo3)ₙ, and reagent grade sodium polysulfonate available from Aldrich Chemicals; tetrasodium salt of 1-hydroxyethane-1,1-diphosphonic acid (HEDP), 21% active, also known as tetrasodium etidronate, available as Briquest ADPA 21 SH; and amino-tri (methylenephosphonate) sodium salt, (ATMP), 30% active, available as Briquest 301-30SH, both available from Albright & Wilson Americas, Glen Allen, Va. The results of this accelerated aging test are given below in Table 2.

TABLE 2
Accelerated Aging of Stabilized Vaterite in water at 120° F.

| | | Aging Time | |
|---|---|---|---|
| Chemical used | Concentration | days | Stability of vaterite |
| None | — | 2 | Complete |
| | | | conversion |
| Sodium | 500 ppm | 21 | Partially converted |
| polyphosphate | | | |
| Sodium | 750 ppm | 21 | No conversion |
| polyphosphate | | | |
| Sodium | 1000 ppm | 21 | No conversion |
| polyphosphate | | | |
| HEDP | 750 ppm | 5 | More than half |
| | | | converted |
| HEDP | 1000 ppm | 7 | Majority converted |
| HEDP | 2000 ppm | 21 | No conversion |
| ATMP | 1000 ppm | 5 | Near complete |
| | | | conversion |
| ATMP | 2000 ppm | 5 | Majority converted |
| Sodium | 2000 ppm | 5 | Complete |
| polysulfonate | | | conversion |

As is seen in Table 2, sodium polyphosphate at a concentration of at least 750 ppm was successful in stabilizing the vaterite product for 21 days; HEDP was successful at a concentration of at least 2000 ppm, while sodium polysulfonate and ATMP were not successful at concentrations up to 2000 ppm. It would have been unexpected to a person of ordinary skill in the art that such improved vaterite stability could be obtained using these stabilizing chemicals at the above-mentioned concentrations.

### EXAMPLE 5

Toothpaste formulations were prepared from inventive Examples 1-2 and Comparative Example 3 to illustrate the advantages of the inventive materials. The amounts of ingredients used to prepare the toothpaste formulations are given in Table 3 below.

The sorbitol and sodium carboxymethyl cellulose are mixed together and stirred until the ingredients are dissolved to form a first admixture. The deionized water, sodium monofluorophosphate, sodium saccharin and sodium silicate are also mixed together and stirred until these ingredients are dissolved to form a second admixture. These two admixtures are then combined with stirring to obtain a “pre-mix”.

The pre-mix is placed in a Ross mixer (Model 130 LDM) and the abrasive vaterite is mixed in without vacuum. A 30-inch vacuum is drawn and the resultant admixture is stirred for approximately 15 minutes. Lastly, sodium lauryl sulfate and flavor are added and the admixture is stirred for approximately 5 minutes at a reduced mixing speed. Toothpaste tubes were filled with the resulting toothpaste, sealed and stored for future evaluations.

Toothpaste compositions containing inventive Examples 1-2 and Comparative Example 3 are given in Table 3 below.

TABLE 3

| | Toothpaste No. | | |
|---|---|---|---|
| Component | 1 | 2 | 3 |
| Sorbitol, 70.0%, g | 23.000 | 23.000 | 23.000 |
| Deionized Water, g | 31.200 | 31.200 | 31.200 |
| CMC-7MXF, g | 1.000 | 1.000 | 1.000 |
| Sodium Saccharin, g | 0.300 | 0.300 | 0.300 |
| Sodium Monofluorophosphate, g | 0.800 | 0.800 | 0.800 |
| Sodium Silicate, g | 0.900 | 0.900 | 0.900 |
| Abrasive | | | |
| Ex. 1 | 40.000 | 0.000 | 0.000 |
| Ex. 2 | 0.000 | 40.00 | 0.000 |
| Comparative Ex. 3 | 0.000 | 0.000 | 40.00 |
| Sodium Lauryl Sulfate, g | 1.800 | 1.800 | 1.800 |
| Flavor, g | 1.000 | 1.000 | 1.000 |

The toothpaste formulations prepared as described above were evaluated for viscosity, RDA and PCR properties according to the following methods with results summarized below in Table 4.

A Brookfield viscometer (Model RVT) with a Helipath stand and spindle T-E was used to determine toothpaste viscosity. The viscometer was set at 5 rpm. The toothpaste sample container was placed in a water bath set at 25° C. to equilibrate. The viscosity was then read at three levels and averaged. Results are reported in centipoise (cps).

RDA was determined according to the article by Grabenstetter, R. J.; Broge, R. W.; Jackson, F. L.; and Radike, A. W.: The Measurement of the Abrasion of Human Teeth by Dentifrice Abrasives: A Test Utilizing Radioactive Teeth, Journal of Dental Research 37: 1060-68, 1958.

The PCR test is described in “In Vitro Removal of Stain With Dentifrice” G. K. Stookey, et al., J. Dental Res., 61, 1236-9, 1982.

TABLE 4
Toothpaste Evaluation

| | | | Viscosity @ 6 wks. |
|---|---|---|---|
| Toothpaste No. | RDA | PCR | Cps |
| 1 | 58 | 108 | 200,000 |
| 2 | 41 | 88 | 360,000 |
| 3 | 153 | 113 | 240,000 |

It is seen in Table 4 that toothpaste made with the inventive products of Examples 1-2 gave excellent cleaning (PCR) results with low abrasion (RDA) values and acceptable thickening properties. The toothpaste containing Comparative Example 3 abrasive had nearly 3 times the RDA value as toothpaste containing the inventive vaterite abrasives. It would have been unexpected to a person of ordinary skill in the art that toothpastes combining excellent PCR cleaning values with low abrasion values could be obtained using calcium carbonate abrasive material.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A vaterite calcium carbonate having a primary particle size of about 0.2 μm to about 3 μm and an aggregate particle size of less than about 4 μm.

2. The calcium carbonate according to claim 1, having a primary particle size of about 0.3 μm to about 2 μm.

3. The calcium carbonate according to claim 1, having an aggregate particle size of less than about 3 μm.

4. The calcium carbonate according to claim 1, having a Brass Einlehner abrasion value of between about 1 mg loss/100,000 rev. to about 5 mg loss/100,000 rev.

5. The calcium carbonate according to claim 1, having a Brass Einlehner abrasion value of between about 1 mg loss/100,000 rev. to about 2 mg loss/100,000 rev.

6. A dentifrice comprising the calcium carbonate of claim 1.

7. A dentifrice comprising from about 30 wt % to about 50 wt % of the calcium carbonate according of claim 1.

8. The dentifrice according to claim 7, wherein the dentifrice has a RDA of about 30 to about 100.

9. The dentifrice according to claim 7, wherein the dentifrice has a PCR of greater than about 80.

10. The dentifrice according to claim 7, wherein the dentifrice has a RDA of about 30 to about 100 and a PCR of greater than about 80.

11. The dentifrice according to claim 7, wherein the dentifrice has a viscosity of less than about 500,000 CPS.

12. A method for forming vaterite calcium carbonate comprising the steps of
(a) preparing a calcium chloride-monoethanolamine solution;
(b) introducing carbon dioxide into the calcium chloride-monoethanolamine solution to form spherical vaterite calcium carbonate by a precipitation reaction; and
(c) adding a stabilizing agent to the spherical vaterite calcium carbonate.

13. The method according to claim 12, wherein step (b) occurs two weeks after step (a).

14. The method according to claim 12, wherein the stabilizing agent is selected from the group comprising sodium polyphosphate and HEDP.

15. The method according to claim 12, wherein about 750 ppm to about 5000 ppm of the stabilizing agent is added.

16. A method for forming stabilized vaterite calcium carbonate comprising the steps of:
forming vaterite calcium carbonate under high shear conditions; and
adding a stabilizing agent to the formed spherical vaterite calcium carbonate.

17. The method according to claim 16, wherein the stabilizing agent is selected from the group comprising sodium polyphosphate and HEDP.

18. The method according to claim 16 wherein about 750 ppm to about 5000 ppm of the stabilizing agent is added.

19. The method of claim 16, wherein greater than about 750 ppm sodium polyphosphate is added

20. A dentifrice comprising the stabilized vaterite according to claim 16.
